(19) United States
(12) Patent Application Publication
(10) Pub. No.: US 2002/0168339 A1
(43) Pub. Date: Nov. 14, 2002

(54) BIOLOGICAL MATERIAL FOR TREATING A MAMMAL BY ANTIBODY GENE TRANSFER AND PHARMACEUTICAL COMPOSITION CONTAINING SAME

(76) Inventors: MARC PIECHACZYK, SAINT GELY DU FESC (FR); DANIELE NOEL, CLAPIERS (FR)

Correspondence Address: IVOR R ELRIFI, MINTZ LEVIN COHN FERRIS GLOVSKY & POPEO, ONE FINANCIAL CENTER, BOSTON, MA, 02111

(21) Appl. No.: 09/341,894

(22) PCT Filed: Jan. 16, 1998
(86) PCT No.: PCT/FR98/00081

(51) Int. Cl. A61K 48/00; C12P 21/04; C07H 21/04; A61K 39/395; C12N 5/06

(52) U.S. Cl. 424/93.2; 424/93.21; 424/130.1; 424/133.1; 435/320.1; 435/326; 435/328; 435/332; 435/339; 435/366; 435/69.1; 435/69.7; 435/69.8; 435/70.1; 514/44; 536/23.53; 536/23.4

(57) ABSTRACT

The invention concerns a biological material for preparing a pharmaceutical composition for treating a mammal by gene transfer, comprising, either at least a nucleic acid sequence containing a therapeutic gene and in a form enabling in vivo transfer of said gene into the cells of the mammal, or at least one cell of the mammal not naturally producing antibodies, genetically modified in vitro by at least a previous nucleic acid sequence, and in a form enabling its incorporation into the organism of the mammal as well as optionally its previous culture. The invention is characterized by the fact that said nucleic acid sequence contains an antibody gene and elements for expressing in vivo said antibody gene and the secretion in the blood circulation of a mammal of a therapeutically effective amount of this antibody or a fragment of it, by cells of said mammal genetically modified by said nucleic acid sequence and not naturally producing antibodies. The invention also concerns the pharmaceutical compositions containing this biological material.

The present invention concerns the area of gene therapy comprised of transferring into the cells of a subject at least one gene coding for a therapeutic protein. More specifically, the invention concerns the transfer, into cells not normally producing antibodies, of nucleic acid sequences coding for all or part or a derivative of therapeutic antibodies involving a protein component participating in the therapeutic effect, so that the cells genetically modified by these nucleic acid sequences and incorporated in a subject produce and secrete in the blood circulation of said subject a therapeutically effective quantity of this antibody.

Gene therapy consists of correcting the deficiency of a gene by introducing into the cells where the deficiency of said gene is the cause of pathology, a DNA sequence carrying the genetic information permitting the deficiency to be remedied. There are numerous areas in which gene therapy may be applied to treat genetic disease; we can cite, for example, correction of thalassemia, sickle cell disease, deficits in liver metabolism, cystic fibrosis, myopathies, etc. (W. F. Anderson, 256, 808, 1992; R. C. Mulligan, Science, 260, 926, 1993; D. Miller, Nature, 357, 455, 1992; R. Morgan and W. F. Anderson, Ann. Rev. Biochem., 62, 191, 1993; B. Dodet, Biofutur, May 1992).

However, gene therapy also permits treating diseases which do not result exclusively from a genetic deficiency, such as cancer or viral infections, by introducing in the cells of the affected organ or tissue a gene coding for a protein or therapeutic RNA. Such therapeutic substances are for example cytokines, intracellular antibodies, viral protein variants, antisense RNA, ribosomes, etc.

The techniques for introducing genetic information into cells are described in the literature. Two major approaches may however be considered.

The first consists of introducing the DNA sequence carrying the genetic information directly in vivo into the cells of the organs or tissues targeted by the therapy

The first consists of introducing the DNA sequence carrying the genetic information directly in vivo into the cells of the organs or tissues targeted by the therapy or in cells of organs or tissues responsible for producing the therapeutic substance, either near its site of production, or systemically.

The second, originating from cellular therapy and termed ex vivo, consists of sampling the cells of a subject, modifying these cells in vitro by introducing the DNA sequence carrying the genetic information to be transferred, and then reintroducing the cells modified in this way into the organism of the subject. This therapeutic strategy is described, for example, in U.S. Pat. No. 5,399,346.

The DNA sequences carrying the genetic information to be introduced into the cells are functionally associated to DNA sequences permitting their expression in vivo and may occur in several forms:

In the case of gene transfer directly in vivo in accordance with the first approach considered above, they may be used:

in free form, or transferred in virgin DNA form, such as a plasmid or restriction fragment, in particular by in vivo injection in the cells, as described in the international patent application published under No. WO 90/11092;

as a complex or associated with other molecules promoting their entry into eucaryotic cells such as lipofectin, transfectase, transfectam, polyethyleneimine, etc.;

incorporated into a viral vector, which is directly introduced in vivo into the cells of the organism or target tissue by infection.

In the case of gene transfer according to the second approach previously considered, termed ex vivo, the DNA sequence is integrated in vitro in the cells which are then introduced into the organism of the subject; it may involve, for example, hematopoietic stem cells, T lymphocytes, hepatocytes, etc. In this case, the cells genetically modified in vitro by the DNA sequence, in accordance with the techniques described above for direct in vivo introduction, may be sampled from the subject treated or come from another human or animal subject such as a pig (E. Cozzi and D. J. G. White, Nature Genetics, 1, 964-966, 1995).

Substances capable of interfering with pathology and which we attempt to produce in the patient's organism for gene therapy include certain antigens or antibodies.

The expression of DNA sequences coding for antigenic proteins aims to permit the production, by cells genetically modified by this DNA, of antigens capable of inducing immunization of the individual. Such a vaccination strategy has, for example, been implemented in the case of different pathogens including the influenza virus (Tang, D., De Vit, M., and Johnston, Nature, 356 152-154, 1992).

The in vitro production of antibodies, fragments of antibodies or derivatives of antibodies such as chimerical antibodies, by genetic engineering in eucaryotic cells has also already been described, for example, in European patents published under numbers 120 694 and 125 023. The injection into patients of therapeutic antibodies aims to target antigens involved in pathology in order to neutralize either directly, or through a chain of metabolic or immune events, one of the causal agents of the disease. Examples of such therapeutic strategies include treatment or prevention of B lymphomas (Yefenof, E., Picker, L. I., Scheuermann, R. N., Vitetta E. S., Street, N. E., Tucker, T., Uhr, J. W., Current Opinion in Immunology, 5, 740-744, 1993).

The international patent application published under No. WO 94/29446 describes the intracellular expression of DNA sequences coding for antibodies. This approach permits considering direct in vivo gene therapy for pathology involving cellular components which are not accessible with traditional vaccination methods or based on the in vivo production of recombinant antigens. The DNA sequences expressed by the genetically modified cells in accordance with the method described in the international patent application WO 94/29446 are therefore essentially characterized by the fact that they include an antibody gene modified so that the antibody is not secreted.

The present invention aims, on the other hand, to implement the in vivo expression of antibody genes by cells which secrete said antibodies in the blood circulation of the mammal carrying the cells genetically modified by the antibody gene.

This invention is based on the demonstration that different cell types, other than those naturally producing antibodies are capable, after genetic modification, of producing antibodies in a stable fashion in vivo.

In fact, the plasmocytes, which are cells specialized for antibody production, are poor candidates for the long-term production of therapeutic antibodies through gene transfer; plasmocytes have a reduced life, on the order of several days, and the fact that they already produce another antibody is likely to lead to associations or recombinations between the antibody chains naturally produced and the antibody expressed by the gene transferred, which is highly damaging to the therapeutic effect sought. It was therefore important to demonstrate that cell types not specialized for the natural production of antibodies were capable of accepting a gene transfer, expressing in vivo a therapeutic antibody and secreting beneficially regulated sustained levels of antibodies in the blood circulation of a mammal.

As a result, the present invention concerns a biological material for preparing a pharmaceutical composition for treating a mammal by gene transfer, comprising,

either at least a nucleic acid sequence containing a therapeutic gene and in a form enabling in vivo transfer of said gene into the cells of the mammal,

or at least one cell of a mammal not naturally producing antibodies, genetically modified in vitro by at least a previous nucleic acid sequence, and in a form enabling its incorporation into the organism of a mammal as well as optionally its previous culture.

Said biological material is characterized by the fact that said nucleic acid sequence contains an antibody gene and elements guaranteeing the in vivo expression of said antibody gene and the secretion in the blood circulation of a mammal of a therapeutically effective amount of this antibody or a fragment of it, by cells of said mammal genetically modified by said nucleic acid sequence and not naturally producing antibodies.

Nucleic acid sequence refers both to DNA and RNA sequences and sequences containing modified nucleotides.

The nucleic acid sequence entering into the composition of the biological material of the invention includes:

at least one therapeutic antibody gene, or a gene coding for a virgin, unmodified, and therefore natural antibody, or antibody fragment, such as Fab or F(ab)₂ or ScFv fragments, or an antibody derivative such as a chimerical antibody or antibody or antibody fragment fused to an effector substance such as a toxin or hormone;

at least one element guaranteeing the expression of the preceding gene; promoter sequences of the transcription placed upstream of the antibody gene and controlling its expression in the cells not naturally producing antibodies.

In addition to the antibody gene and its promoter, the nucleic acid sequence can include a termination sequence of the transcription, situated downstream from the antibody gene and permitting the secretion of antibody gene product in the blood circulation of the mammal some of whose cells have been genetically modified by the nucleic acid sequence.

The promoter used may be any promoter permitting an effective expression of the gene it controls in the cell type genetically modified by the nucleic acid sequence. It may therefore be a viral promoter, a ubiquitous or specific tissue promoter or a synthetic promoter.

According to a first potential embodiment of the invention, the biological material would include a nucleic acid sequence containing an antibody gene and elements guaranteeing the in vivo expression of said antibody gene and the secretion in the blood circulation of a mammal of a therapeutically effective quantity of this antibody or a fragment of the latter, by cells of said mammal genetically modified by said nucleic acid sequence and not naturally producing antibodies, with said sequence in the form of a virgin DNA or RNA sequence. More specifically, virgin DNA sequence refers to a plasmid, but may also be any other form of DNA such as a restriction fragment. A pharmaceutical composition based on this biological material may be administered to an individual by local injection or electroporation; it then contains, in addition, the nucleic acid sequence(s) of the biological material, a pharmaceutically acceptable vehicle or adjuvant compatible with nucleic acids.

According to a second potential embodiment of the invention, the biological material would include a nucleic acid sequence containing an antibody gene and elements guaranteeing the in vivo expression of said antibody gene and the secretion in the blood circulation of a mammal of a therapeutically effective quantity of this antibody or a fragment of the latter, by cells of said mammal genetically modified by said nucleic acid sequence and not naturally producing antibodies, with said sequence in the form of a complex or conjugated with a molecule or carrier substance promoting its penetration in the target cells, such as liposomes or lipid vesicles.

According to a third potential embodiment of the invention, the biological material would include a nucleic acid sequence containing an antibody gene and elements guaranteeing the in vivo expression of said antibody gene and the secretion in the blood circulation of a mammal of a therapeutically effective quantity of this antibody or a fragment of the latter, by cells of said mammal genetically modified by said nucleic acid sequence and not naturally producing antibodies, with said sequence in the form of a transfer vector. The vector in which the antibody gene is incorporated may be a biological viral vector, such as a retrovirus, an adenovirus, a parvovirus or any other vector permitting the effective transfer in vivo of the antibody gene in the cells of a mammal. A pharmaceutical composition based on this biological material may be administered to an individual either locally or systemically in accordance with traditional gene transfer methods, by transfection of DNA or RNA or infection by a virus.

A fourth potential embodiment of the invention derives from a gene transfer strategy through cellular therapy incorporating genetically modified cells. In this potential embodiment, the biological material of the invention would be comprised of cells not naturally producing antibodies, and in a form permitting them to be incorporated in the organism of a mammal as well as optionally its previous culture, said cells being genetically modified by at least one nucleic acid sequence containing an antibody gene and elements guaranteeing the in vivo expression of said antibody gene and the secretion in the blood circulation of a mammal of a therapeutically effective quantity of this antibody or a fragment of the latter.

The preceding embodiment of the invention has two variants:

The cells not naturally producing antibodies entering into the composition of the biological material of the invention may derive from the mammal to be treated. In this variant, the cells are prepared using the traditional techniques of cellular and molecular biology, such as for example from biopsies taken from the patient to be treated. These cells are then genetically modified by the nucleic acid sequence carrying the antibody gene, either by transfection or infection with a vector conforming to those described above in the case of a direct in vivo gene transfer. Pharmaceutical compositions manufactured from this biological material are given back to the patient from whom the cells have been sampled.

The cells not naturally producing antibodies entering into the composition of the biological material of the invention may derive from a human or animal mammal other than the one to be treated. These cells were prepared as in the preceding variant. In the case of cells of human origin, these cells come from compatible donors; in the case of cells of non-human origin, cells of genetically modified animals are used, such as the pig, made compatible for an organ transplant.

The preceding cells are in a form permitting their incorporation by any known method in the organism of the receiving mammal. They may, in addition, be in a form which has enabled them to be cultured prior to the graft. It may be comprised of any culture substrate or medium compatible with their administration and incorporation in the receiver, such as a matrix of the type described in the European patent application published under No. 378 576 concerning fibroblasts.

In the fourth potential embodiment, as well as for the preparation of nucleic acids entering into the composition of biological materials of other potential embodiments of the invention, cells are chosen which do not naturally produce antibodies but which do have:

the ability to be able to secrete proteins in the blood circulation of a mammal;

a long life in the mammal's organism, preferably at least several months to several years up until the entire lifetime of the patient.

More specifically, for the fourth potential embodiment of the invention, these cells are selected for their capacity to easily tolerate being sampled, genetically modified ex vivo and incorporated into a mammal.

Among the cell types having the preceding characteristics, the invention relates more specifically to keratinocytes, hepatocytes, skin fibroblasts, myoblasts, endothelial cells and hematopoietic stem cells.

It has surprisingly been demonstrated (Fenjves, E. S., Smith, J., Zaradic, S., and Teichman, L. B., Human Gene Therapy, 5, 1241-1248, 1994) that the keratinocytes could relatively effectively produce proteins for the organism and not only for external use. In addition, they have been easily and routinely cultured for several years in hospital departments for skin grafts.

Hepatocytes are more difficult to handle than keratinocytes. However, it has been demonstrated (Grossman, M., Raper, S. E., Kozarsky, K., Stein, E. A., Engelhart, J. F., Muller, D., Lupien, P. J., Wilson, J. M., Nature Genetics, 6, 335-341, 1994; Ferry, N., Duplessis, O., Houssin D., Danos, O., Heard J-M., Proc. Natl. Acad. Sci., USA, 88, 8377-8381, 1991) that hepatocytes can be infected by recombinant retroviruses both ex vivo and in vivo.

Retroviral culture and transduction of skin fibroblasts is easy to do (Moullier, P., Marechal, V., Danos, O., Heard, J-M., Transplantation, 56, 427-432, 1993). Organoids are easy to handle (Moulier et al, Nature Genetics, 4, June 1993), 154-159). Fibroblasts have the advantage of being easy to sample in a subject by using a simple surgical procedure. In addition, gene therapy protocols are underway to correct lysosomal deficits in children.

Myoblasts, which are undifferentiated muscle cells, can also be purified, and will likely be used without genetic modification for the treatment of certain degenerative diseases (Yao, S-N., Smith, K. J., and Kurachi, K., Gene Therapy, 1, 99-107, 1994).

Genetic modification of endothelial cells has already been done to produce therapeutic proteins, for example in PCT international patent application published under No. WO 90/06997. The endothelial cells, which comprise the blood vessel walls, are therefore specifically adapted to the incorporation of the biological material of the invention, the goal of which is to cause the genetically modified cells to secrete antibodies into the blood circulation of a mammal.

Other cell types may be considered, such as hematopoietic stem cells, if they have the characteristics defined above.

The biological material of the invention is used in the preparation of pharmaceutical compositions for the treatment or prevention of cancer relapse, and viral infection or spread, AIDS in particular.

One out of four people is affected by cancer in the Western world and the treatments available today are truly satisfactory for only about one out of two.

Serious viral diseases are increasingly affecting human populations, in particular, of course, the AIDS virus, for which there currently is no effective means of preventing or treating infection.

The biological material of the invention is significant in that it permits us to consider a new therapeutic approach for these very serious diseases.

In fact, in the case of cancer, it would permit the organism to use over the long-term specific antibodies of tumor cells, either cytocides, or those inducing cellular dormancy. This goal is reached in using nucleic acid sequences carrying a gene coding for antibodies directed against a specific tumor cell antigen.

In the case of viral infections, the biological material of the invention permits the organism to maintain over the long term a base antibody level which is either neutralizing for the virus, or cytocide for the infected cells. This goal is attained using DNA sequences coding for antibodies directed against a specific antigen of the virus responsible for said infection or against a specific antigen of the cells infected by said virus.

The present invention also concerns, therefore, the pharmaceutical compositions comprising a biological material such as that defined above. In addition to the biological material of the invention, these compositions may contain traditionally used vehicles or adjuvants. The doses of biological material entering into these pharmaceutical compositions are adapted to the method of administration used, the pathology targeted, the nucleic acid sequence implemented and its form of presentation to permit the production and secretion of a therapeutically effective quantity of antibody in the blood circulation of the subject treated.

The invention also concerns human or non-human cells not naturally producing antibodies, but genetically modified by at least one nucleic acid sequence containing a therapeutic antibody gene and elements guaranteeing the in vivo expression of said antibody gene and the secretion into the blood circulation of a mammal which has received said cells of a therapeutically effective quantity of this antibody or a fragment of it.

These cells comprise a biological material which is specially adapted for the preparation of pharmaceutical compositions for cellular therapy ex vivo of an individual.

The preceding human cells, when they do not originate from the patient in whom they are implanted, or animal cells, are, of course, prior to implantation, treated by all physical or genetic methods known in the field so that they are protected from the immune system of the patient receiving them.

The invention also concerns the use of a biological material or preceding cells, for the preparation of a pharmaceutical composition for the treatment of cancer or viral infections. The invention also concerns the use of a nucleic acid sequence containing an antibody gene and elements guaranteeing the expression in vivo of said antibody gene and the secretion in the blood circulation of a mammal of a therapeutically effective quantity of this antibody or a fragment of it, by cells of said mammal genetically modified by said nucleic acid sequence and not naturally producing antibodies, for the preparation of pharmaceutical compositions for treating mammals by gene transfer. More specifically, this use targets the preparation of pharmaceutical compositions for treating cancer or viral infections.

Lastly, the invention concerns a manufacturing process for a cell genetically modified by at least one DNA sequence coding for a therapeutic antibody or fragment of this antibody, characterized by the fact that DNA sequences coding for a therapeutic antibody or a fragment of this antibody are transferred by any appropriate method, into cells not naturally producing antibodies, and by the fact that cells genetically modified by said DNA sequence are selected from these cells.

In addition to the preceding characteristics, the invention includes other characteristics which will appear in the course of the following description and which refer to experimental examples of embodiment and implementation of the present invention, it being understood that these examples do not in any way constitute a limitation to the scope of the claims.

The work reported below permitted demonstration of the following:

in vitro, cells which may be sampled from the patient, genetically modifiable ex vivo and reimplantable, not naturally producing antibodies, are capable of secreting recombinant antibodies retaining the properties of the original antibody,

at least one preceding cell type is capable of secreting in vivo recombinant antibodies retaining the properties of the original antibody.

the biological material of the invention does not induce any immune response neutralizing the recombinant antibody in the modified organism.

I-Definition of the Recombinant Antibody

The model recombinant antibody used for the antibody gene transfer experiments cited below is a mouse anti-human thyroglobulin monoclonal antibody (Tg10) (Piechaczyk et al., Hybridoma, vol. 4, 4, (1985), 361-367). Its molecular cloning and functional characterization of the complementary DNA of its heavy chain and its light chain were carried out as indicated below.

Thyroglobulin is an iodinated glycoprotein of high molecular weight involved in the synthesis, storage and secretion of the T3 and T4 thyroid hormones (Marriq, C., C. Arnaud, M. Rolland, and S. Lissitzsky, 1980, Eur. J. Biochem. 111 3347). A mouse monoclonal antibody, hereinafter referred to as Tg10, directed against an antigenic region (region II) frequently recognized by natural autoantibodies in patients with Grave's disease, Hashimoto's thyroiditis and thyroid carcinoma, was established by CNRS UMR 9921 laboratory members of the Faculte de pharmacie de Montpellier (Montpellier Faculty of Pharmacy), Avenue Charles Flahaut, 34060, Montpellier Cedex 01, France (Peichaczyk et al., Hybridoma, vol. 4, 4, (1985), 361-367). The complementary DNA of the light chains (Kappa) and heavy chains (IgG₁) of the Tg10 antibody were cloned in the pSPORT1 vector (Gibco/BRL) with traditional techniques of genetic engineering (Sambrook, J., E. F. Fritsch, and T. Maniatis, 1989, Molecular Cloning: A Laboratory Manual. Cold Spring Harbor Laboratory Press, Cold Spring Harbor, N.Y., USA). The nucleotide sequences of the variable parts of the light and heavy chains which specify each of the antibody chains were determined and are represented respectively in FIGS. 1 and 2 in the appendix. cDNA cloning and sequencing of the TglO antibody were performed in the laboratory mentioned above.

For their functional characterization, the cDNA of the light chain and heavy chain of antibody Tg10 were cloned in the pLXPXSN retroviral vector (Morgan, R. A., L. Couture, O. Elroy-Stein, J. Ragheb, B. Moss, and W. F. Anderson, 1992. Nucl. Acids Res 20, 1293-1299) either on both sides of the IRES sequence of the poliovirus endogenous to this vector to form the PM130 vectors, or individually upstream of the IRES sequence to form the PM117 and PM124 vectors, as represented in FIG. 3 in the appendix. The simian cells COS-7 (ATCC CRL 1651) were then transfected with the calcium phosphate technique or with PM130 alone, or with the PM117 +PM124 combination. The presence in the culture supematants of antibodies reactive against human thyroglobulin was tested with the ELISA technique (Piechaczyk et al., Hybridoma, vol. 4, 4 (1985), 361-367). In addition, kinetic association and dissociation constants of the Tg10 recombinant antibody produced by the COS-7 cells with thyroglobulin were determined from culture supernatants with surface plasma resonance (Fagerstam, L. G., and R. Karlsson. 1993. Biosensor techniques. In Immunochemistry. V. Oss and M. VanRegenmortel, eds. M. Dekker Inc. p. 949-970) according to the Biacore technique developed by Pharmaci Biosensor Company.

The values of these constants are reported in TABLE 1 below.

TABLE 1

| | Kinetic association | Kinetic dissociation | |
|---|---|---|---|
| Antibody | constant kₒₙ (M⁻¹S⁻¹) | constant k_{off} (s − 1) | Affinity constant Kₐ |
| Natural Tg10 | 4.6 ± 0.1 × 10⁵ | 5.3 ± 0.2 × 10⁻⁵ | 8.6 ± 0.4 × 10⁹ |
| antibody | | | |
| Recombinant Tg10 | 1.4 ± 0.3 × 10⁵ | 4.3 ± 1.0 × 10⁻⁵ | 3.2 ± 1.4 × 10⁹ |
| antibody (PM117 ± | | | |
| PM124) | | | |
| Recombinant Tg10 | 2.1 ± 1.5 × 10⁵ | 6.0 ± 0.4 × 10⁻⁵ | 3.5 ± 2.7 × 10⁹ |
| antibody (PM130) | | | |
| Heavy chain of the | 1.0 ± 0.3 × 10⁵ | 3.0 ± 0.2 × 10⁻⁴ | 3.3 ± 1.2 × 10⁸ |
| recombinant Tg10 | | | |
| antibody (PM117 ± | | | |
| PM124) | | | |

Surprisingly, TABLE 1 demonstrates that the heavy chain synthesized alone from PM124 is secreted by the COS-7 cells and recognizes human thyroglobulin with an affinity decreased only tenfold relative to the complete antibody.

II - Retrovirus Generating Lines

Most of the primary cells are extremely sensitive to traditional transfection methods. In addition, the length of life, and therefore expression, of transfected DNA is generally very short in most transfected cells.

To permit an effective infection of varied cellular types and long-term expression of the Tg10 antibody in the genetically modified cells, a cellular line generating recombinant retroviruses transporting and expressing cDNA of the Tg10 antibody was established.

The PA 317 cells with amphotropic retroviral packaging (Miller, D. and Buttimore, 1986, Molec. Cell. Biol. 6, 2895-2902) were transfected with the calcium phosphate precipitate technique with retroviral vector PM130. Several stable producer clones were established. The PA1 30.10 line was used for the later infection experiments. Its virus titer, dosed on the NIH 3T3 indicator line (Miller, D. and Buttimore, 1986, Molec. Cell. Biol. 6, 2895-2902) was 10⁴ cfu/ml.

III - In vitro Experiments

The retroviruses produced by the PA130.10 line were used to infect different established cell lines representative of different cell types available from the American Type Culture Collection (ATCC):

NIH3T3 murine fibroblast line;

A431 human keratinocyte line;

HepG2 human hepatocyte line;

C2C12 myoblast line.

Different cellular clones were derived for each type of retroviral transduction and the Tg10 antibody produced in the culture supernatant was dosed using ELISA. The results obtained are reported in TABLE 2 below.

TABLE 2

| Lines | Tg10 antibodies |
|---|---|
| NIH3T3 line | 88 +/− 65 ng /10⁵ cells/24 hours |
| A431 line | 35 +/− 6 ng/10⁵ cells/24 hours |
| HepG2 line | 3.5 +/− 1.5 ng/10⁵ cells/24 hours |
| C2C12 line | 2 +/− 0.6 ng/10⁵ cells/24 hours |

In addition, in the case of C2C12 myoblasts differentiated in vitro in myotubes, production is retained.

The thermodynamic and kinetic properties of antibodies produced by these different cell types determined by surface plasma resonance according to the BlAcore technique (Pharmacia Biosensor) proved to be identical to those of the initial Tg10 antibody.

Retroviral vectors were then used to infect primary mouse skin fibroblasts (human retroviral infection and hepatocytes (transfection). The antibody production was respectively:

10 to 20 ng/10⁵ cells/3 days, and

1 to 10 ng/10⁵ cells /4 days.

Likewise, the characteristics of the antibodies produced were the same as those of the initial antibody.

IV - In Vivo Experiment

C2C12 cells genetically modified and which retained the capacity to differentiate into myotubes were implanted via injection in the forelegs of 4 syngenic C3H mice on the basis of 10⁷ cells per foreleg.

In 3 of 4 mice, the production of recombinant antibodies having retained the thermodynamic properties, and the recognition property of the initial antibody antigen was followed for two months. The quantity of antibody produced was regularly elevated from the base level to a production of approximately 100 ng/ml of serum.

V-Absence of Immune Response Neutralizing the Recombinant Antibody

One of the essential goals of the invention is to systemically produce a recombinant antibody, beneficially therapeutic by genetically modified mammalian cells.

A possible risk of this approach is the induction of an immune response on the part of the modified organism capable of neutralizing the recombinant antibody.

This potential problem was avoided in the experimental results presented below. 2×10⁷ primary myogenic cells expressing a stable Tg10 monoclonal antibody after retroviral transduction were implanted at the level of the tibialis anterior of the C3H mouse. The mouse serum was sampled at one week intervals for several months. The quantity of Tg10 antibody secreted was dosed with the ELISA method. In parallel, the quantity of anti-idiotype antibody was determined by ELISA.

In a series of 5 mice, secretion of Tg10 antibody was between 100 and 300 ng/ml of serum for 4 months. No anti-idiotype response could be detected under these conditions.

1. Biological material for preparing pharmaceutical compositions for treating a mammal by gene transfer, comprising, either at least a nucleic acid sequence containing a therapeutic gene and in a form enabling in vivo transfer of said gene into the cells of the mammal, or at least one cell of the mammal not naturally producing antibodies, genetically modified in vitro by at least a previous nucleic acid sequence, and in a form enabling its incorporation into the mammal's organism as well as optionally its previous culture. The invention is characterized by the fact that said nucleic acid sequence contains an antibody gene and elements for expressing in vivo said antibody gene and the secretion in the blood circulation of a mammal of a therapeutically effective amount of this antibody or a fragment of it, by cells of said mammal genetically modified by said nucleic acid sequence and not naturally producing antibodies.

2. Biological material according to claim 1, characterized by the fact that it includes a nucleic acid sequence containing an antibody gene and elements for expressing in vivo said antibody gene and the secretion in the blood circulation of a mammal of a therapeutically effective amount of this antibody or a fragment of it, by cells of said mammal genetically modified by said nucleic acid sequence and not naturally producing antibodies. This sequence is in the form of a virgin DNA or RNA sequence.

3. Biological material according to claim 1, characterized by the fact that it includes a nucleic acid sequence containing an antibody gene and elements for expressing in vivo said antibody gene and the secretion in the blood circulation of a mammal of a therapeutically effective amount of this antibody or a fragment of it, by cells of said mammal genetically modified by said nucleic acid sequence and not naturally producing antibodies. This sequence is a complex or is conjugated with a molecule or carrier substance.

4. Biological material according to claim 1, characterized by the fact that it includes a nucleic acid sequence containing an antibody gene and elements for expressing in vivo said antibody gene and the secretion in the blood circulation of a mammal of a therapeutically effective amount of this antibody or a fragment of it, by cells of said mammal genetically modified by said nucleic acid sequence and not naturally producing antibodies. This sequence is a vector permitting the effective transfer in vivo of the antibody gene in cells.

5. Biological material according to claim 4, characterized by the fact that the vector is a biological viral vector.

6. Biological material according to claim 1, characterized by the fact that it is comprised of cells not naturally producing antibodies, in a form which permits their incorporation into the mammal's organism as well as optionally its previous culture. Said cells are genetically modified by at least one nucleic acid sequence containing an antibody gene and elements for expressing in vivo said antibody gene and the secretion in the blood circulation of a mammal of a therapeutically effective amount of this antibody or a fragment of it.

7. Biological material for the preparation of pharmaceutical compositions for the treatment of mammals with gene transfer, according to claim 6, characterized by the fact that the cells not naturally producing antibodies come from the mammal to be treated.

8. Biological material for the preparation of pharmaceutical compositions for the treatment of mammals with gene transfer, according to claim 6, characterized by the fact that the cells not naturally producing antibodies come from another mammal than the one to be treated and have undergone treatment making them compatible.

9. Biological material according to any of the preceding claims, characterized by the fact that the cells not naturally producing antibodies are selected from those which have:
the ability to be able to secrete proteins in the blood circulation of a mammal;
a long life in the mammal's organism.

10. Biological material according to claim 9, characterized by the fact that the cells not naturally producing antibodies are selected from those which may be easily be sampled, genetically modified ex vivo and implanted in a mammal.

11. Biological material according to any of claims 9 and 10, characterized by the fact that the cells not naturally producing antibodies are selected from keratinocytes, hepatocytes, skin fibroblasts, myoblasts, endothelial cells and hematopoietic stem cells.

12. Biological material according to any of the preceding claims, characterized by the fact that the antibody gene is a gene coding for a virgin antibody, fragment or derivative of this antibody such as a chimerical antibody.

13. Biological material for the preparation of pharmaceutical compositions for the treatment or prevention of cancer in a subject, in accordance with claim 12, characterized by the fact that said antibody, fragment or antibody derivative is directed against a specific tumor cell antigen.

14. Biological material for the preparation of pharmaceutical compositions for the treatment or prevention of an infection or viral expansion in a subject, according to claim 12, characterized by the fact that said antibody fragment or antibody derivative is directed against a specific antigen of the virus responsible for said infection or against a specific antigen of cells infected by said virus.

15. Pharmaceutical composition comprised of a biological material according to any of claims 1 to 14 preferably associated with a pharmaceutically acceptable vehicle.

16. A human or non-human cell not naturally producing antibodies, characterized by the fact that it is genetically modified by at least a nucleic acid sequence containing a therapeutic antibody gene and elements guaranteeing the expression in vivo of said antibody gene and the secretion in the blood circulation of a mammal having received said cells of a therapeutically effective quantity of this antibody or a fragment of it.

17. Use of a biological material according to any of claims 1 to 14 or cells according to claim 16, for the preparation of a pharmaceutical composition for the treatment of cancer or viral infections.

18. Use of a nucleic acid sequence containing an antibody gene and elements guaranteeing the expression in vivo of said antibody gene and the secretion in the blood circulation of a mammal of a therapeutically effective quantity of this antibody or a fragment of it. The cells of said mammal genetically modified by said nucleic acid sequence and not naturally producing antibodies, for the preparation of pharmaceutical compositions for the treatment of mammals with gene transfer.

19. Use according to claim 18, for the preparation of a pharmaceutical composition for the treatment of cancer or viral infections.

20. Manufacturing process for a cell according to claim 16, characterized by the fact that using any appropriate method, one transfers at least one nucleic acid sequence containing an antibody gene and elements guaranteeing the expression in vivo of said antibody gene and the secretion in the blood circulation of a mammal of a therapeutically effective quantity of this antibody or a fragment of it, by cells of said mammal genetically modified by said nucleic acid sequence and not producing antibodies naturally in cells not naturally producing antibodies, and by the fact that those cells genetically modified by said nucleic acid sequence are chosen from among these cells.
